(19) United States
(12) Patent Application Publication
(10) Pub. No.: US 2010/0268300 A1
(43) Pub. Date: Oct. 21, 2010

(54) LOW FREQUENCY ELECTRICAL STIMULATOR DEVICE FOR THE PREVENTION AND TREATMENT OF CHRONIC WOUNDS

(75) Inventors: Gabriela Ramos Leal, Guadalajara Jalisco (MX); Sergio Antonio Mendoza Zepeda, Guadalajara Jalisco (MX)

Correspondence Address: KNOBBE MARTENS OLSON & BEAR LLP, 2040 MAIN STREET, FOURTEENTH FLOOR, IRVINE, CA, 92614, US

(73) Assignees: Gabriela Ramos Leal, Guadalajara, Jalisco (MX); Sergio Antonio Mendoza Zepeda, Guadalajara, Jalisco (MX); Carlos Arturo Plasencia Moeller, Zapopan, Jalisco (MX); José Fernando de Jesus Plasencia Moeller, Zapopan, Jalisco (MX); José Fernando Plasencia Nuñez, Zapopan, Jalisco (MX)

(21) Appl. No.: 12/601,249

(22) PCT Filed: May 21, 2007
(86) PCT No.: PCT/MX07/00063

§ 371 Date: Jun. 25, 2010

(51) Int. Cl. A61N 1/00

(52) U.S. Cl. 607/50

(57) ABSTRACT

A low frequency electrical stimulation device for the prevention and healing of chronic wounds resulting from diabetes, or other type of pathology. This device applies electrical stimulation through a square bipolar signal, and a paused squared bipolar signal in at least one of the output channels, and at different therapy times and frequencies that will depend upon the specific needs of the patient.

### FIELD OF THE INVENTION

This invention is targeted to the medical field, specifically for the treatment of patients with chronic wounds stemming from diseases such as diabetes, that are receiving treatment in hospitals, rehabilitation or personal use. The invention consists of an electrical stimulator device that treats the surrounding tissue of a wound for the regeneration of the area that presents the ulcer wounds.

### BACKGROUND OF THE INVENTION

One of the most feared problems that affect the quality of life of diabetics is a foot ulcer. The foot is particularly vulnerable to circulatory and neurological damages, any minor trauma can cause pain or infection and these can ultimately lead to amputation of limbs.

Great part of the problem of diabetic foot complications is associated with individual factors, and with medical service factors (7):
The inadequate therapeutic regimen and lack of therapy compliance of the diabetic patient, in conjunction with the lack of modification of eating habits, with little or no family involvement.
The lack of specialized centers that specifically treat the diabetic foot problem, involving an interdisciplinary team composed of: family members, nutrition specialist, social worker, nurse, psychologist, family doctor, surgeon, orthopedist, angiologist, internist, endocrinologist, and rehabilitation physician.
The lack of availability of time from the family doctor during control consults, since patient education regarding foot problems requires more time than a routine consult.
Insufficient knowledge of the warning signs of diabetic food and the preventive measures to decrease the incidence of serious problems.
The lack of importance given to the patient's work habits, like risk on work to suffer a trauma or standing for long periods of time.

Now days, new techniques and devices have been developed to help in the healing of wounds that work under the electrotherapy principle. Electrical stimulation is defined as the set of techniques that apply electrical current to the body with different therapeutic objectives. This is done through special devices that transfer an electrical current to the body using two or more electrodes that are applied directly on the skin, which are able to go through the skin layers, the subcutaneous cell tissue and the intended nerve or muscle fiber, so biological and physiological reactions can be produced, thus improving the different tissues when they are affected by different diseases or metabolical disorders which would cause skin ulcers in the limbs.

It has been proven that the electrical stimulus is effective to enhance the healing rate for patients with diabetic foot and skin ulcers. Low frequency impulse improves the circulation and the tissue nutrition, as well as accelerates formation of granulation tissue, increases capillary density and oxygen levels (14).

Wound healing through electrical stimulation is attributed to the increment in blood flow. The electrical current impulse theory proposes that this response is moderated by the electrical stimulation of the peripheral nervous system.

In U.S. Pat. No. 4,233,965 (Fairbanks) a device and a method are proposed to treat skin diseases or traumas, which is based on applying monopolar square stimuli or ramp stimuli to the skin via electrodes. It is a two channel analog device, and its function principle is based on two oscillatory circuits, two voltage multipliers and a ramp signal generator. These circuits are in charge of obtaining the electrical impulse that is applied to the patient.

A method for treating wounds through electrical stimulation is described in U.S. Pat. No. 5,158,081; through a device that uses three kinds of square modified DC waveforms (bipolar), and applies them mainly to bedsores or ischemic ulcers.

On the other hand, other similar devices such as the one described in European patent EP1374946, describes an electrical stimulator for muscle conditioning and aesthetic treatments. It is a digital system, it provides monopolar stimuli adjusted in the current, and it has a feedback system detector of voltage peaks and temperature sensors. It also uses an analog to digital converter (ADC) and an amplifier that are all integrated in the CPU, and also it has an acoustic alarm. Another device of similar characteristics is described in U.S. Pat. No. 4,913,148, which is used to treat zoster herpes.

Other similar devices, like the one described in U.S. Pat. No. 5,063,929, uses the same principle of transcutaneus nerve stimulation through symmetrical biphasic impulses. It also has a unit control that generates the impulses and it has channels that control or adjust the current that the patient receives. One of this device's disadvantages is that it uses more electronic components to generate the bipolar signals, such as an analog digital converter or multiplexers.

In general, electrotherapy devices are of low or medium frequency and have a wide spectrum of frequency ranges, voltages and currents. It should be noted that the majority have luminous and sound displays that provide the information to the user regarding the type of signal, frequency and warnings. However, there are differences that characterize each device; for example, and to mention some of the ones that are present in this invention, and that will be described in detail in the following pages, are: in the way that the electrical impulses are generated by a microcontroller; the type of impulses such as square bipolar wave form and paused square bipolar wave form; the configuration of four independent channels of the electrical stimulator, which makes it useful clinically due to the fact that two to four different patients can receive customized therapy treatments, giving the doctor or therapist in charge of the therapy a lot more flexibility or in its personal use mode in which the device can be used in an easy and quick manner.

The independence of channels is an inconvenience in the stimulators that have been analyzed, because in clinical use, it makes therapy treatments more complicated since each patient has different specific needs.

With the aim of eliminating these inconveniences and to improve other characteristics of low frequency electrical stimulators that are currently used, the present stimulator was conceived in its two variants that we will describe later on, for the prevention and healing of chronic wounds caused by diabetes or other diseases.

### AIM OF THE INVENTION

The first objective of the present invention, it's to provide an innovative device to assist the healing process of chronic skin ulcers in patients caused by diseases such as diabetes and/or venous insufficiency. This would be achieved by applying a combination of electrical stimulation of the square bipolar waveform and paused square bipolar waveform.

The second objective of the invention is to propose a new method in the treatment for prevention and healing of wounds or chronic skin ulcers in a patient, by applying an electrical stimulation procedure with two types of different electrical impulses in the affected area.

These two objectives, and other aims of the invention, are materialized as a new electrical low frequency stimulator device for the prevention and healing of chronic wounds, such as:

A) A control board consisting of:
i) a microcontroller that generates electrical impulses in the form of a square monopolar wave signal and a paused square monopolar wave signal in at least one output channel at different frequencies. The microcontroller generates different time bases for the application of therapy and also provides to each output channel one of the aforementioned frequencies and signal type according to the therapeutic needs of the patient;
ii) at least one signal conditioning circuit which is sensitive to any current variation provided by the signals from outputs of the microcontroller, the signal conditioning circuit would provide two signals 180° out of phase with respect to the microcontroller;
iii) at least one controlling and amplifier circuit powered by a voltage supply constituted by opto-transistors and complementary transistors, of which two are of the NPN type and two more are of the PNP type, in an H bridge configuration. The two signals coming out of the signal conditioning circuit activate the opto-transistors and these opto-transistors activate a branch of the H bridge, and based on the phase of the signal conditioning circuit, the bipolar signals are obtained in its two waveforms on the signal amplifier output;
iv) at least one channel indicator circuit to activate a bicolor light emitting diode that is activated depending on the polarization voltages in one of the microcontroller outputs;
v) an oscillatory circuit to set the speed at which the microcontroller executes the instructions stored in its memory, and said oscillatory circuit includes a crystal whose value may change according to the type of controller or speed required;
vi) a pre-establishing circuit to allow the microcontroller to re-establish its system in a manual form, or an automatic form as the device is turned on; and
vii) a regulated power supply to power the circuit and the low frequency electrical stimulator device for the prevention and healing of chronic wounds.

### BRIEF DESCRIPTION OF THE ATTACHED DRAWINGS

FIG. 1 depicts the first section of the electrical diagram of control board's design, which is the low frequency electrical stimulator device for the prevention and healing of chronic wounds. It should be noted that the interconnection between the circuits, in the diagrams, is done through tags that point out the inputs and outputs of each circuit or block.

FIG. 1A depicts the second section of the electrical diagram's design in FIG. 1.

FIG. 1B illustrates the third section of the design of the electrical diagram of the previous FIG. 1.

FIG. 2 is a top view of the low frequency electrical stimulator device for the prevention and healing of chronic wounds.

FIG. 2A is a top view of the electrical stimulator device, in its pre-established therapy version.

FIG. 2B is a frontal perspective view of the electrical stimulator device, in its pre-established therapy version.

FIG. 3 is a top view that shows the cables and electrodes that are applied topically and used to transfer the electrical impulses of the electrical stimulator device to patient's skin.

FIG. 4 is a rear view that exemplifies the low frequency electrical stimulator device of this invention.

FIGS. 5 and 5B are lateral views, right and left respectively, of the low frequency electrical stimulator device for the prevention and healing of chronic wounds.

FIG. 6 is a graphic representation of the square monopolar waveform generated by the electrical device's microcontroller.

FIG. 6A is a graphic representation of the paused square monopolar waveform generated by the microcontroller.

FIG. 7 is a graphic representation of the square bipolar waveform generated by the signal conditioning circuit.

FIG. 7A is an image depicting the paused square bipolar waveform generated by the signal conditioning circuit.

FIG. 8 is a comparative graphic of the patient's sensitivity before and after the treatment.

FIG. 9 is a comparative graph of the patient's level of pain before and after the treatment.

### DETAILED DESCRIPTION OF THE PREFERRED EMBODIMENT

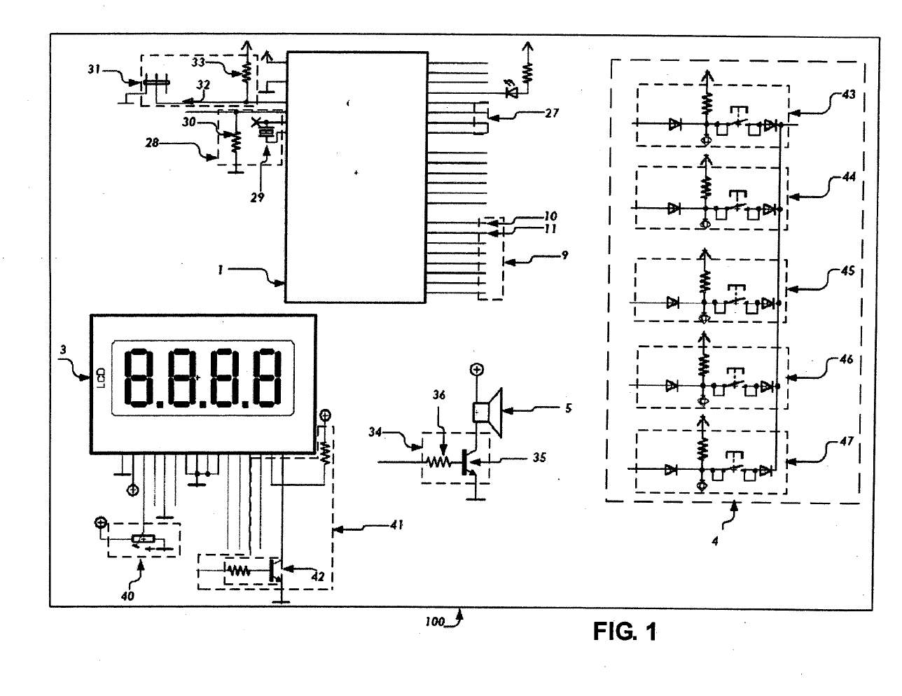

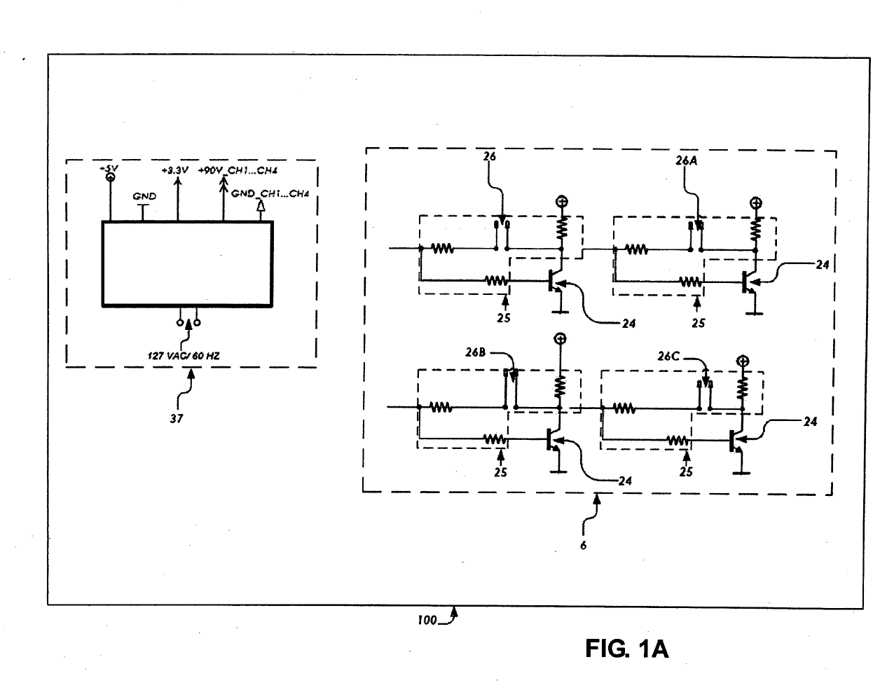

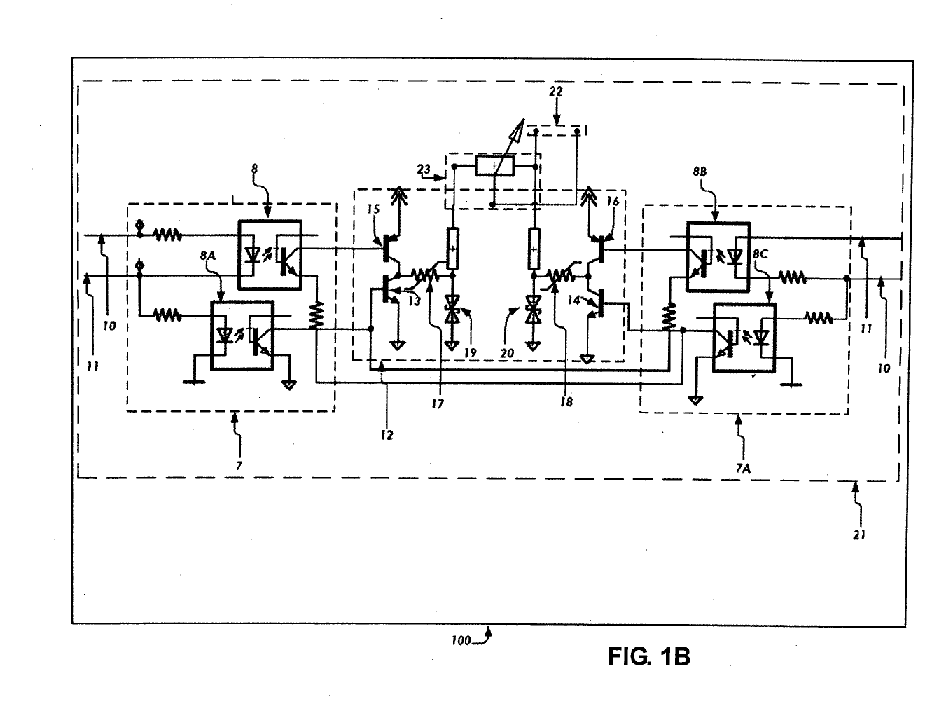

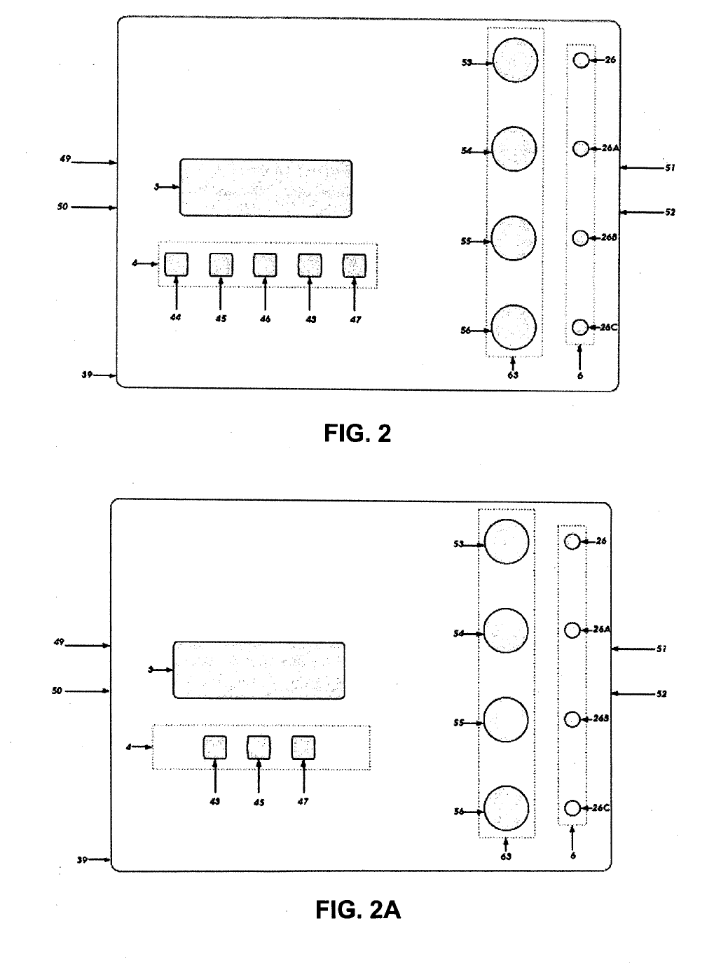

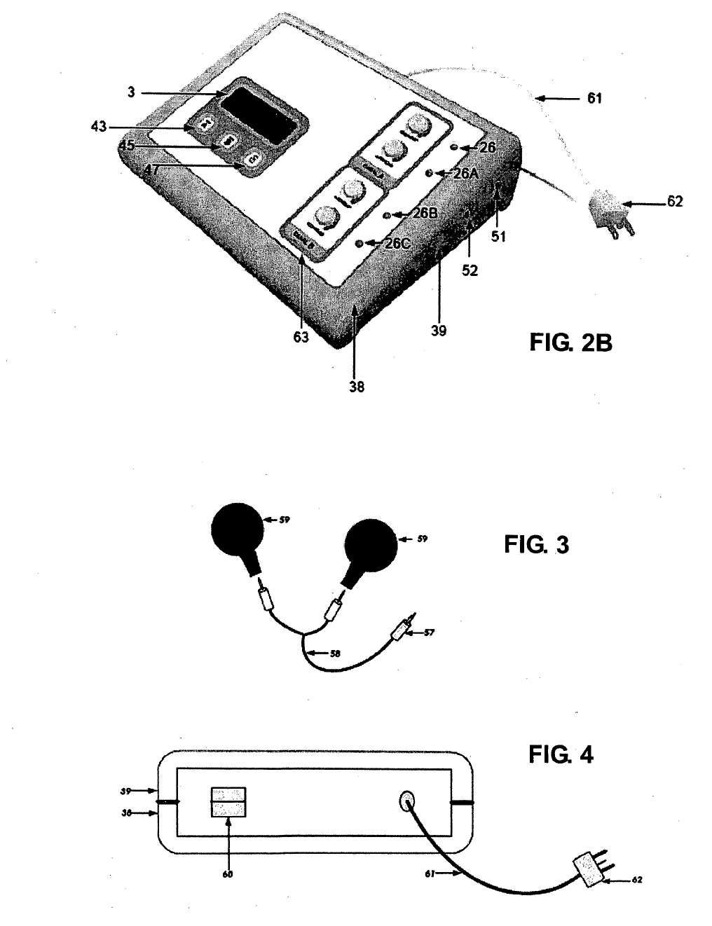

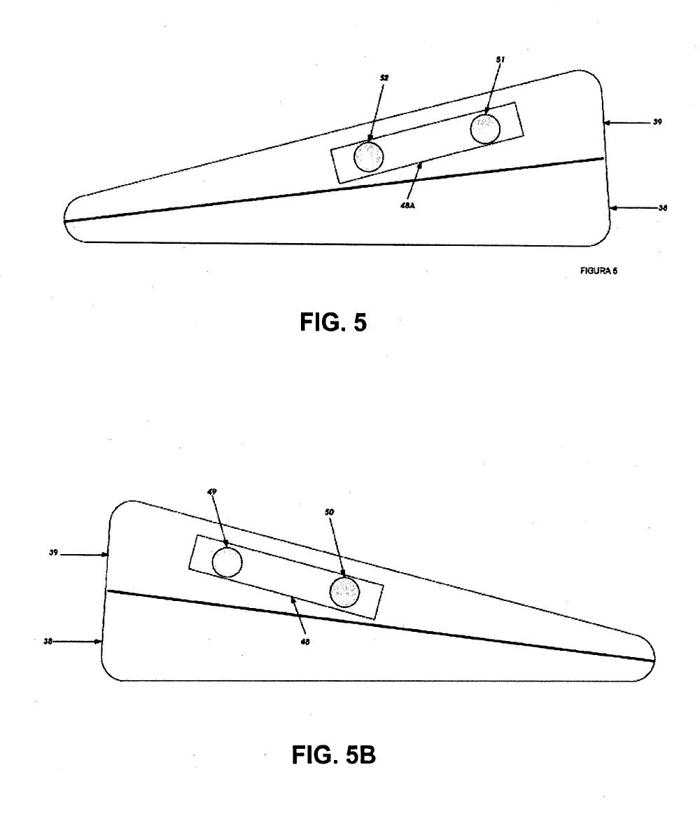

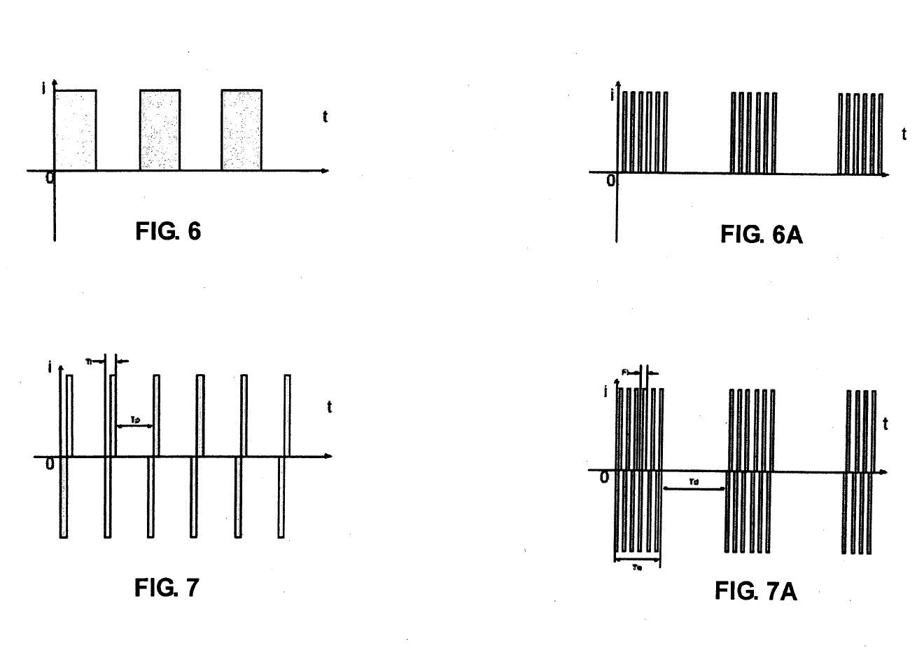

Regarding these accompanying images, the low frequency electrical stimulator device for the prevention and healing of chronic wounds, in a first mode of embodiment is constituted mainly by:
A) A control board (100) that consists of:
   i) a microcontroller (1) that generates electrical impulses in the form of a monopolar square wave signal (FIG. 6) and a paused square monopolar wave signal (FIG. 6A) in at least one output channel (48, 48A) at frequencies of 50, 100, 150, 200 and 250 Hz. The microcontroller (1) generates time bases for the implementation of therapies of 5, 10, 15 and 30 minutes and also provides for each output channel (49, 50, 51 and 52) one of the frequencies and type of signal, square monopolar wave signal and paused square monopolar wave signal, as required by the therapeutic needs of the patient. Also, the microcontroller (1) configures and controls: a graphic display (3) although this element may not exist, a keyboard (4), a sound alarm (5) and channel indicator circuits (6). The frequency ranges, therapy times and types of signals may be conveniently adjusted according to the therapeutic needs. The use of the microcontroller (1) has the advantage that it avoids the use of electronic components such as multiplexors, external memories, and digital analog converters, thus reducing the size and complexity of the electrical stimulators. It also has the advantage of providing more independence in the management of the parameters of the signal in each one of the output channels;
   ii) at least one signal conditioning circuit (7 and 7A) made up of 4 optotransistors (8, 8A, 8B, 8C) and three resistors that make the circuit sensitive to a voltage variation, the inputs (10, 11) of the signal conditioning circuits (7 and 7A) are the signals coming from outputs of (9) the microcontroller (1). Two pins are used in the microcontroller (1) for each output channel. The output channel provides the square monopolar waveform (FIG. 6) and paused square monopolar waveform (FIG. 6A), and the signals between the two pins (10, 11) of the microcontroller (1) are out of phase 180°;
   iii) at least one control and amplifier circuit, powered by a power supply (37) of +90V, made up of 4 complimentary transistors of which two are of the NPN type (13, 14), and two more of the PNP type (15, 16), in an H bridge configuration. The signals (10, 11) coming from the microcontroller activate the opto-transistors (8, 8A, 8B, 8C), and these have the capacity of activating a branch of the H bridge (14, 15) or (13, 16), according to the negative or positive pulse of the signals to be formed; this allows to generate the bipolar signals in both of their forms (FIGS. 7 and 7A). To be able to guarantee the stipulated voltage and current requirements, mainly for the user's protection, two very important elements are included in the transistor's outlet: fuses (17, 18) that are placed in series for the protection of current exceeding those stipulated in the design or an energy overloading, and varistors (19, 20) placed in parallel to protect against abrupt voltage variations or surges. The control and amplifier circuit (12) in addition with the signal conditioning circuits (7, 7A) make up the potency circuit (21), and at least one of them would be in the electronic board, and the amount of these would depend on the number of channels that the electrical stimulator device requires. For each power circuit (21) there is an outlet (22), which is regulated by a lineal potentiometer (23) with a switch, and these circuits (21) are connected to one of the outlets of the electrical stimulator device (49, 50, 51, 52);
   iv) at least one channel indicator circuit (6) made up of an NPN transistor (24) and three resistors (25) which configure the transistor as a not logic gate. The function of the indicator circuit is to activate a bi-color emitting diode (26, 26A, 26B, 26C) depending on the polarization voltages in the microcontroller outlets (27). The purpose of these circuits (6) is to provide more information to the user regarding the operation of the device, such as clarifying the type of impulse that is being applied and to protect the patient by indicating the activated channels;
   v) an oscillatory circuit (28) (FIG. 1) consisting of a crystal (29) and a resistor (30) which set the speed at which the microcontroller (1) executes the instructions stored in its flash memory. The crystal value may change depending on the type of microcontroller or the speed required;
   vi) a pre-establishing circuit (31) to allow the microcontroller (1) to re-establish its system in a manual form, or an automatic form as the device is turned on. The pre-establishing signal comes from one microcontroller (1) output (32) which is connected to a pull-up resistor (33) which closes the circuit through the pre-set jumper;
   vii) a current amplifier circuit (34) made up of a BJT NPN transistor (35) and a resistor (36) limiting the current which drives the sound alarm (5) when the microcontroller (1) issues the command; and
   viii) a regulated power supply (37) of: +3.3V, +5V and +90V@ 40 mA that powers the potency circuit (21) and provides energy to the electrical stimulator device. This circuit has an internal fuse that limits the current to no more than two amps.
B) A protective housing, composed of two parts (FIGS. 2, 4, and 5) in which we can observe a base (38) and an upper cover (39), where we will find the following elements:
C) A graphic display (3), where the electrical stimulator's configuration and operation menus can be visualized. The display has a contrast circuit (40) made up of by a voltage driver, for controlling the background light, and “on/off” switch (42). The circuits (41) and (42) are optional, depending on the type of graphic display used. The display itself may not be necessary for an electrical stimulator that provides recorded therapy programs;
D) at least one external EEPROM or FLASH memory that allows the storage of pre-defined programs or configurations of the device. This is an optional item;
E) at least one RS-232 interface to be able to communicate with a device that supports this protocol. This is an optional item;
F) a keyboard (4) to access the menu and the configurations of the electrical stimulator device, which would include:
   a “start” button (43) used to enable the channels, the clock, and to start the therapy.
   a “function” button (44) that shows the following options to configure:
      1. Number of channels to be used.
      2. Individual Channel Configuration (waveform and signal frequency)
      3. Length of therapy.
   A “selection” button (45) to display the different options for each menu.
   An “accept” (46) button to choose the desired parameters, and record the chosen parameter.
   A “stop” button (47) that will disable the channels being used and activates the alarm, allowing re-start the therapy immediately, without having to configure the parameters again, by only pressing the “start” button.
      In a second form of embodiment of the electrical stimulator device: the pre-established therapy (FIG. 2A); the keyboard is constituted by the following buttons:
   a button (43) with a square bipolar signal mode: in the channel outlets (48, 48A) the signal depicted in FIG. 7 is shown.
   a button (45) for paused square bipolar signal: in the channel outlets (48, 48A) the signal in FIG. 7A is depicted.
   a “stop” button (47): that will disable the channels being used and activates the alarm, allowing to re-start the therapy immediately, without having to configure the parameters again, by only pressing the “start” button.
G) at least one potentiometer (23) connected as a voltage divider in the H bridge circuit outlet (12). This potentiometer regulates the voltage applied to the monophonic connectors (48, 48A) (FIG. 5) which constitute each one of the outlet channels (49,50,51,52);
H) at least one knob (63) placed on the upper housing (39), which is assembled into a potentiometer (23) which is able to adjust the intensity in each of the output channels [49,50,51,52] in a more user friendly manner;
I) at least one monophonic output (48, 48A), where a male type plug (57) (FIG. 3) is joined with the electrode cable (58) through which the signals (FIGS. 7 and 7A) will be transmitted to the electrodes (59) and finally to the patient;
J) a “on/off switch” (60) directly connected to the cable (61) to turn the device “on or off”; and
K) a cable (61) with plug (62) to accept an alternating current of 127V, 60 Hz AC, with 3 shunts to supply electric energy to the regulated power source (37).

### Method of Operation of the Invention

To operate the low frequency electrical stimulator device for the prevention and healing of chronic wounds correctly, it is necessary to follow these steps:
A) connect the device to a 127V/60 Hz plug;
B) place switch (60) in an “on” position;
C) watch the “Welcome” Message on the graphic display (3) and wait for the message “Configuring Channels”;
D) the device will wait until the FUNCTION key (44) is pressed;
E) the channels will be configured with the following buttons: SELECTION (45) to choose an option, and ACCEPT [46] to record the petition. For each channel, the following parameters must be selected:
   a) Active channels to be used.
   b) Choose the type of signal and frequency for each channel.
   c) Store length of therapy.
F) once the channels have been configured, a pre-defined message shows on the display (3) and the indicators (6) will be red;
G) then introduce the connector (57) to the channel to be used (49, 50, 51, 52) and also connect the cable (58) to the electrode pairs (59);
H) then the electrodes are placed on the patient longitudinally in the direction of the nerves near the wound. In this case the polarity of the electrodes does not matter, because the signals are not polarized;
I) the device waits for the START (43) button or the FUNCTION (44) button to be pressed:
   a) FUNCTION: please see parenthesis E.
   b) START: verify that the operation parameters are programmed, if they are not; please go back to parenthesis E.
      i. displays the warning sign: “turn knobs off”
      ii. The device will begin to generate electrical impulses (FIG. 7 and/or FIG. 7A) into the selected channels, (49, 50, 51, 52), will activate the channel indicators (6) and will start to operate the therapy clock, and this will show on the display (3). In the beginning, the intensity must be adjusted to a comfortable level for the patient, and this is done through the knobs (53, 54, 55,56)
      iii. In the meantime, the device waits for the end of the established time period or to be interrupted by the user through the STOP button (47).
         1. STOP button (47): the signal transmittal will stop, the channel indicators being used will be deactivated (6), the alarm will be activated (5) for 3 seconds, and the previously recorded parameters will be re-established.
         2. Time up: The generation of the signal has ended, channel (6) indicators are deactivated, the alarm (5) is activated for seven seconds and the parameters previously recorded are reset.
      iv. Finally the electrodes are removed from the patient, the device status returns to point E or the switch (60) can be turned off.

In the case of the pre-established therapy version, the steps (C) (D) (E) and (I, section a) in the above processes are omitted.

### Effect of the Electrical Stimulator Device for the Treatment of Vascular Lesions and/or Diabetic Foot

The main objective of this user study is to evaluate the clinical effects that are produced by the combination of square bipolar electrical signals and paused square bipolar electrical signals in the treatment of chronic wounds caused by diabetes and/or venous insufficiency. For this purpose, 27 patients were selected who had skin ulcer lesions in their lower extremities, the lesions were evolved over at least one month and a maximum of 40 years. During the treatment, the ulcers were evaluated qualitatively and quantitatively to monitor the response to the applied electrical stimuli. The data was subjected to parametric statistics and non parametric statistics.

There was one group selected at random which also served as a control group. The patients that were accepted to participate in the user study, had to meet the following criteria to be included: individuals with more than one ulcer with a grade 1-3 lesion¹ caused by diabetes complications and peripheral vascular insufficiency that were monitored during their chronic illnesses, such as diabetes mellitus and arterial hypertension, under the established criteria. ¹ Wagner classification

Subjects that were excluded from the user study were those for whom electrical stimuli was not recommended for the following reasons: (1) cardiac arrhythmias, (2) use of a pacemaker, (3) active thrombophlebitis, (4) osteosynthesis material such as nails, staples, screws, etc. (5) pregnant women, (6) individuals with osteomyelitis. Also, those individuals that in the beginning of the user study show the following conditions: (7) infected wounds, (8) gangrene of a limb, (9) being intolerant to electrical stimuli, (10) complications that warrant hospitalization during the investigation, (11) missing more than 5 appointments in a month, (12) changes in their state of awareness, judgment or mental condition, (13) did not want to continue the user study after signing a letter of informed consent.

### Patient's Demographics

The information of the patients that were enrolled in the user study was obtained through a Clinical History (Table I). There were a total of 13 women and 14 men registered; with an average age of 67.15 years (SD=10.36, range=34-85). The average time of evolution time of the ulcers was 89.89 months (SD=135.91, range=1-480). The ulcer with the longest evolution time was 40 years. The majority of the ulcers were located between the knee and the ankle; 13 of the 35 ulcers were located between the ankle and the back of the foot, the rest were on the toes or the sole of the foot.

The origin of the treated ulcers was mainly pressure, neuropathy and trauma, combined with venous and arterial insufficiency. Also, the patients had diseases such as: diabetes mellitus, arterial hypertension, diabetes mellitus/systemic arterial hypertension (DM/SAH) in combination with venous or arterial insufficiency.

TABLE I
Demographics of patients and medical conditions
coexisting in the initial evaluation

| | X̅ | S.D. | Range |
|---|---|---|---|
| Demographics | | | |
| Age | 67.15 years | 10.36 | 34-85 years |
| Ulcer evolution time | 131.69 months | 159.46 | 1-480 months |
| Size of ulcer | | | |
| Length | 92.55 mm | 243.02 | 8-1500 mm |
| Width | 37.42 mm | 55.70 | 6-350 mm |
| Depth | 6.89 mm | 4.45 | 2-30 mm |

| Cause of the ulcer | Number of ulcers |
|---|---|
| Pressure | 4 |
| Neuropathic | 6 |
| Traumatism | 9 |
| Venous insufficiency and/or | 26 |
| arterial | |
| Diseases that caused | |
| the ulcer | No. of subjects |
| Diabetes Mellitus | 5 |
| D.M./S.A.H. | 4 |
| Arterial Hypertension | 5 |
| Peripheral venous | 13 |
| insufficiency | |

X̅ Statistical Mean
S.D. Standard Deviation

### Data Analysis

The evaluation of the demographics and medical conditions of the individuals were obtained via an interview and with direct observation of the patients, such as: age, ulcer evolution time, degenerative chronic diseases, glucose control, and blood flow.

The analysis was performed to determine the difference between the size of the wound at the beginning and the end of the user study, was through a Student's T test for parametric data such as width, length and depth. In the case of non parametric data, such as the healing rate of each individual, the change in sensitivity and pain was done using the Fisher Exact Test and χ2. In both tests, the analysis was made of the whole group and another was a classification of the individuals by etiologies (diabetic patients, hypertension patients, diabetic/hypertension patients, and venous insufficiency patients).

The acceptance criterion to determine the statistical significance of the data were the values whose probability (p) was higher than 0.05.

### Initial Evaluation

The information on the initial status of the patient was obtained from questionnaires that allowed the elaboration of a clinical history during the user study development. In these questionnaires the habits of the patient were verified, risk factors were determined, qualitative and quantitative ulcer characteristics were registered and an initial photograph of the wound was recorded on a form.

The information gathered was regarding the main diseases of the patient, glucose control, nutritional control, weight control, medications administered, exercise program, type of movement (whether it was dependant or independent). There was also a review of personal hygiene habits and feet cleaning habits such as cutting and trimming of toenails. There was an identification of risk factors such as proper footwear, addictions or signs of hypo or hyper glycemia. On the other hand, low feet temperature was verified, as well as pain, claudication, epithelialization, change in muscle form (keloid scarring) and presence of pedal pulse. In the skin check up, exudate amount was observed, as well as skin texture, skin hydration and lubrication, fissures, wounds, skin color, infections, alterations in sensitivity, and finally there was a picture taken to register the appearance, classification, grade, exudate type and size of the ulcer.

During the user study, it was attempted not to change patient's behavior and personal habits, unless it was necessary to preserve their health. Weight control, blood pressure (BP) and glucose testing was done through their family doctor in their monthly control consults, and evaluation for pain and sensitivity was done through pain questionnaire. Skin temperature, edema, pain tolerance, pedal pulses, skin appearance and ulcer condition was done every 15 days through the medical evaluation of the physician in charge of the program.

### User Study Design

There was a quasi-experimental, randomized and prospective investigation in which 27 patients met the criteria to be included, and formed the control group to which the electrical stimulation therapy was applied. It is worth noting that the same control group was used to compare the electrotherapy results versus other alternative treatments that the same patients had used before start the present user study.

The group was treated for a one-year period applying electrical stimulation with the electrical stimulator device of the present invention, twice a week for a period of 30 minutes, 15 minutes with the square bipolar signal and 15 minutes with the paused square bipolar signal. After the therapy, wound care followed, such as: wound cleansing, dressing and/or bandage changing. In subjects with more than one ulcer, electrodes were applied in such a way that the electrical impulses were received in the whole affected area. Among these cases, there were 6 individuals with two wounds on one foot, and one with two ulcers on each leg. The subjects whose wounds were totally healed through the course of the treatment (nine people in the group) the electrical stimulation was applied as a preventive way for another 15 days, none of those nine patients came back to treat a recurrent ulcer.

The patients were instructed that during, and in the final phase of the sessions, they should not feel any kind of discomfort; the sessions would be comfortable, in the form of a massage, with probably just an itching sensation around the ulcer. This itching sensation would have to disappear 15 minutes after the therapy. They were also informed of the fact that any symptom of pain, cramping, or irritation should be reported to the nurse to adjust the electrical stimulating device or to advise the healthcare professional if the therapy should be stopped. None of the individuals reported discomfort or a reaction caused by the stimulation.

In the biweekly evaluations the doctor in charge conducted a general inspection of the wounds and debridement procedures, were performed if the ulcers that developed necrotic or sphacelus tissue that impeded skin regeneration warranted it.

Generally speaking, the necrotic tissue or the sphacelus tissue was developed around the ulcer, and in all subjects the development of granulation tissue from the bottom of the wound to the skin surface was observed; that is, the healing of the wound started by healing the depth of the ulcer, and then the length and width of the ulcer. In some cases, there were symptoms of infection identified so an antibiotic was prescribed by the family doctor.

### Treatment Sessions

The programming of the electrical stimulator device that was used for the user study was: 15 minutes of square bipolar signal, and 15 minutes of paused square bipolar signal, at a 100 Hz and a maximum intensity of 200V (this was always regulated according to the patient's tolerance and sensitivity). The patients received these therapies for a period of 30 minutes twice a week.

The placement of the electrodes was done in a way that the impulses surrounded the wound, on healthy skin, and avoiding contact with wounds, fissures, thick or scaly skin.

The polarization or electrode order of the electrodes was not relevant, because the currents are not polarized (biphasic).

The cleansing of the wound was always closely watched and controlled by the healthcare professional in charge of the user study, any infection symptom or anomaly was supervised by the nurse in charge at the time of the wound cleansing, or by the caregivers in charge of cleaning the patients taking part in the user study.

All the materials/substances applied on the wounds were previously sterilized, and when the electrode sessions were over, the cables and electrodes were cleaned with an antiseptic solution and soap. During the cleaning and revisions of the wounds, there were precautions taken at all times to use disposable materials/substances for cleaning the wounds as well as hand washing, latex gloves, and decontamination of the area and equipment used.

### Evaluation During Treatment

The doctor in charge reported, every fifteen days, new measurements and aspect of the wound: necrotic plaque, sphacelus, granulation tissue, epithelization, type of exudate (serous or purulent) and signs of infection (purulent exudate, red edge wound, bad odor, torpid evolution). Besides, all changes observed in the patient were registered, such as: medication (oral and topical), pain symptoms, or discomfort of the patient during therapy. When complications arose, such as cellulitis or necrobiosis, the patient was attended by competent medical personnel.

On the other hand, measurements were taken of the width, length, and depth of the wound, and if there was an internal and external diameter of the cellulitis these measurements were also obtained; these measurements were the basis to assess a wound's status. The measurements were taken with a vernier caliper (with an accuracy of tenths of mm) and there were sketches of the wound drawn in the file regarding the reference used for these measurements. The rate of decrease in the wound area was calculated for each one of the patients at the begging and at the end of the user study.

Finally, pictures were taken of the different leg perspectives, which allowed for a full view of the wound that was treated.

### Results

Comparing wound measurements at the beginning and at the end of the user study, we found that in each and every one of the groups there was a significant change in the three dimensions: (width, χ²=22.12, p=0.0000026; length, χ²=17.72, p=0.0000256; depth, χ²=26.97, p=0.0000002).

It is important to point out that initially, wounds that measured more than 10 mm. in length and width reduced its size up to 50%, the final measurement of the wounds being between 0 and 9 mm. As far as depth is concerned, there was a change of approximately 61.11%.

TABLE II
Comparison of the size of the ulcer at the beginning and end of the user
study.

| | |
|---|---|
| Ulcers size between | |
| 0 y 9 mm | Ulcers size of more 10 mm |

Width; χ² = 22.12, p = 0.0000026

| | | |
|---|---|---|
| Beginning of the User | 8.33% | 91.67% |
| Study | | |
| End of the User Study | 61.11% | 38.89% |

Length; χ² = 17.72, p = 0.0000256

| | | |
|---|---|---|
| Beginning of the User | 5.56% | 94.44% |
| Study | | |
| End of the User Study | 50.00% | 50.00% |

Depth; χ² = 26.97, p = 0.0000002

| | Ulcers size between | |
|---|---|---|
| | 0-2 mm | Ulcers size of more 3 mm |
| Beginning of the User | 16.67% | 83.33% |
| Study | | |
| End of the User Study | 77.78% | 22.22% |

The first changes that were observed was the reduction in the wound's depth, the wound being covered with granulation tissue, and once the granulation tissue reached the top of the wound, the wound started to close up in its width and length. In two of the cases, there was no closure in length and depth due to the chronic nature of the wound; however the surface of the wound reached the level of the normal skin.

Dividing the patients by etiology, the Student's T test showed that there was only a significant change in diabetics and in patients suffering from venous insufficiency.

TABLE III
Comparison of the size of ulcers stratified by etiology.

| | Before | | After | | | |
|---|---|---|---|---|---|---|
| Variable | X̅ [mm] | S | X̅ [mm] | S | Student's T | P |

Diabetes Mellitus

| | | | | | | |
|---|---|---|---|---|---|---|
| Width | 18.667 | 5.055 | 1.000 | 2.236 | 5.430 | 0.0001444 |
| Length | 30.667 | 3.249 | 1.333 | 2.981 | 11.302 | 2.56 × 10⁻⁷ |
| Depth | 5.333 | 2.285 | 0.500 | 0.764 | 3.408 | 0.003339 |

Venous insufficiency

| | | | | | | |
|---|---|---|---|---|---|---|
| Width | 30.200 | 19.884 | 17.050 | 20.534 | 1.128 | 0.1332 |
| Length | 122.650 | 317.719 | 84.400 | 257.603 | 0.229 | 0.4099 |
| Depth | 7.250 | 5.647 | 2.800 | 2.657 | 1.748 | 0.0443 |

Hypertension

| | | | | | | |
|---|---|---|---|---|---|---|
| Width | 36.429 | 20.653 | 26.286 | 23.939 | 0.574 | 0.2881 |
| Length | 100.429 | 89.497 | 77.571 | 95.796 | 0.312 | 0.380 |
| Depth | 5.286 | 1.278 | 2.286 | 2.491 | 1.919 | 0.039 |

Hypertension/Diabetes Mellitus

| | | | | | | |
|---|---|---|---|---|---|---|
| Width | 13.250 | 5.540 | 5.250 | 2.947 | 1.857 | 0.05635 |
| Length | 16.250 | 6.647 | 7.750 | 2.861 | 1.711 | 0.06900 |
| Depth | 6.500 | 3.775 | 4.250 | 2.861 | 0.692 | 0.25745 |

All Group

| | | | | | | |
|---|---|---|---|---|---|---|
| Width | 37.417 | 55.700 | 14.472 | 18.110 | 1.125 | 0.1321 |
| Length | 94.083 | 243.090 | 28.111 | 52.600 | 0.762 | 0.2243 |
| Depth | 6.9440 | 4.530 | 2.556 | 2.550 | 2.772 | 0.0035 |

It should be noted that in this particular test, the standard deviation was very wide, due to data dispersion [Table I].

The group was divided according to the time in which the ulcer had not healed, to determine the healing rate in each patient.

It was observed that the patients which had had an ulcer for less than 2 years, had a healing rate between 80% and 100%; and individuals with wounds that had been there for more than 25 months did not reach this level of healing. However, there was a change or significant healing rate of 1% to 79%.

In cases where there was no healing (chronic cases) it is important to point out that there was no case that showed regression in the wound, and at least the wound did not get any larger in size.

TABLE IV
Healing rate in the size of the ulcer according to evolution time.

| | | |
|---|---|---|
| | Ulcers with 1 to 24 | Ulcers with more than 25 |
| % of Healing | months of evolution | months of evolution |

Width; χ² = 13.81, p = 0.001004

| | | |
|---|---|---|
| 80%-100% | 67% | 0% |
| 1%-79% | 17% | 89% |
| 0% | 17% | 11% |

Length; χ² = 14.54, p = 0.000696

| | | |
|---|---|---|
| 80%-100% | 61% | 0% |
| 1%-79% | 22% | 100% |
| 0% | 17% | 0% |

Depth; χ² = 2.71, p = 0.2577

| | | |
|---|---|---|
| 80%-100% | 56 % | 22% |
| 1%-79% | 39% | 67% |
| 0% | 6% | 11% |

During the year in which the electrical stimulation was applied, 37% of patients had complete 100% healing (χ²=4.43, p=0.0363) even though this result was achieved only in patients that had had the wound for less than 2 years. This was not the case in any other group.

During the sessions, (6-8 weeks) remarkable changes were observed, such as change in skin color (a reduction in rusty red coloration) and the appearance began to change to a healthy skin tone. In other patients, the temperature in lower extremities improved, and in the majority of the cases there was an improvement in pain symptoms, parastesias, tingling sensation and there was an increase in pain tolerance. In others, the edema and the exudates decreased. The main changes in the healing of the wound were observed in the first 15 days of therapy.

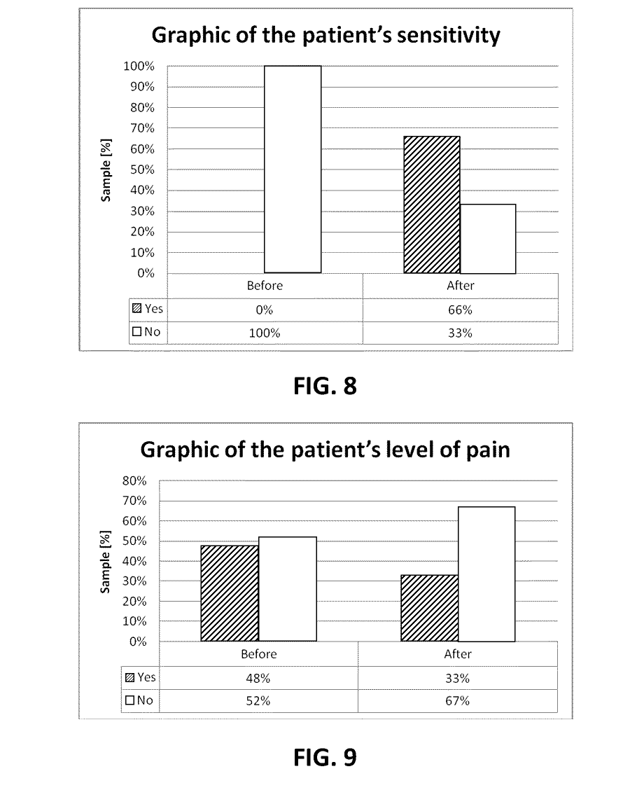

The change in sensitivity was measured in a very discrete way: normal sensitivity or a change in sensitivity. A change in sensitivity was noted in patients that presented a combination of factors such as neuropathy, angiopathy, intrinsic and extrinsic pressures originated by bone deformities in the foot [2]. At the beginning of the user study, the totality of the patients showed neuropathy symptoms or loss on sensitivity to cold, after the first week of therapy patients expressed an increased sensibility to the cold and lessening of numbness and cramps. According to Fisher's Exact Test, remarkably 66% of the patients (p=0.0016) recovered sensitivity in the damaged extremities (FIG. 8).

As far as pain reduction, statistically there were no significant changes in patients (X2=1.23, p=0.26) since there was almost the same rate of people that had had pain or no pain at the end of the user study (FIG. 9).

### General Conclusions

Through this user study, it was demonstrated that applying electrical stimulation to skin ulcers (diabetes, trauma, venous insufficiency and or arterial insufficiency) according to the previously described protocol, accelerates the healing process since in 67% of the patients the surface of the wound was reduced by more than 50%.

On the other hand, it was proven that applying electrical stimulation is not a painful therapy and it is a safe procedure for the patients. It is also considered to be a treatment with little or no side effects [3, 4], and it is also a method that only requires very simple training for the nurses, therapists and physicians.

Even though the user study was designed for a very small sample, it was enough to detect clinically significant differences.

In this user study, all factors that were thought to affect the healing of the ulcer were monitored and registered, such as: the demographics of the subjects, medications applied and ulcer care. Among these factors, there was no difference detected, that is why it is believed that the acceleration of the healing process was due to the electrical stimulation applied in the above described terms.

The benefits of the therapies were more noticeable in young people or in people that had wounds that were less than two years, or those in which the disease (such as diabetes and hypertension) was not that advanced. There was also a finding that the healing is affected by the lack of control in glucose levels, especially in cases of hyperglycemia.

It was also proven that the electrical stimuli applied in the treatment and with the device here described, is effective for the enhancement of the healing rate for patients with ulcers [3] because it improves circulation and tissue nutrition, it accelerates the formation of granulation tissue, it also enhances capillary density, and partial oxygen pressure [4]. The electrical current theory proposes that this response is mediated through the electrical stimuli of the peripheral nervous system [5] thus causing the release of neurotransmitters in the peripheral extremities of this nerves (Levine, J. D.).

The physiological basis consists in stimulating the nerve endings to provoke the venous and lymphatic vasoconstrictor reflex, and the motor response to the flat muscle that is found in the stimulated area, accelerating drainage of retained fluids, due to trophism and tissue damage caused by the ulceration.

According to the studies that were done, it was proven that electrical stimulation can be used for the rehabilitation of the capillarity system and some nerves, in such a way that damaged areas (ulcers) will get stimulated, and this will lead to creating new synapses, carry oxygen and nutrients that will re-generate damaged tissue. It was also demonstrated that the application of the electrical stimuli on people that suffer from chronic wounds caused by peripheral circulation deficiencies, would help improve blood flow, and this will accelerate the healing process.

In this user study, the electrical stimulator device was used to apply continuous bipolar square wave signals and paused bipolar square wave signals, since the human nervous systems generates pulses or peaks of triangular current, and this way the stimulators must get responses that the nervous system is not able to generate. The device must also provide symmetrically compensated waveforms to avoid an electrical stimuli sensation, skin irritation and burns.

It is important to take into account that only a very small amount of energy is needed to obtain efficient stimuli. In the user study the level of intensity that was used was comfortable for the patient, and it also increased circulatory response due to the fact that intense or painful stimulation, or vigorous activation of the muscular contraction, may diminish blood flow [5].

Even though the two therapies per week option was used, it is foreseen that the frequency of the therapies can be reasonably incremented, and this would reduce the size of the ulcer at a faster rate [6].

The best performance in the application of electrical therapy in diabetic foot ulcer with circulatory damages in the extremities was observed in ulcers of thromboembolic nature, in which there was peripheral vascular insufficiency. However, more prospective studies are required to be able to more accurately assess the electrical therapy technique for ulcer management, especially in cases in which ulcers have been in place for more than 15 years, and other types of pathologies, for example, Charcot's Neuropathy.

Another feature of this invention consists in a treatment method for patients that have skin ulcers stemming from a degenerative disease such diabetes. The method consists of stimulation of the sensory nerve endings to provoke the venous and lymphatic vasoconstrictor reflex and a motor response from the flat muscle that is found in the stimulated area, thus accelerating drainage of retained fluids, due to trophism and tissue damage caused by the ulceration. The electrical stimulation consists of applying electrical impulses, near the wound and in alternate sessions of 15 minutes each of a square bipolar signal and a paused square bipolar signal, at 100 Hz per time period in the signal, and a maximum intensity of 200V.

### Final Conclusions

The realization of this user study applying the electrical stimulator device and method of this invention would allow us to conclude that:
The patients will have a better evolution if the therapies are applied more than two times per week, even every day of the week as long as it does not exceed 30 minutes so it will not cause muscle fatigue.
It helps as support therapy in the treatment of ulcers caused mainly by diabetic foot or venous insufficiency.
It can be applied to any type of ulcer.
It improves circulation and tissue nutrition, it accelerates the growth of granulation tissue, and it enhances capillary density and partial oxygen pressure.
It can be used as a preventive method.

Even though the device and the method have been described in the context of their preferred modality or forms of embodiment, for specialists in this area, it will be evident that the scope of the concept can reach beyond the design specifically described and illustrated to other possible alternative embodiments of the invention that are feasible or viable. Furthermore, although the invention has been described in detail and any expert in this field can reach the conclusion that some of the elements that constitute the device can be substituted and others can be incorporated to the above mentioned description without modifying the essence of it, and the way it operates as well as the result for which it has been created.

Taking all of this into account, it will be understood that various elements of the device and method, can be combined with others, or be substituted by others to conform the alternate ways in which the device can function that would lead to the same result for which it has been created. Therefore, it is the intention that the reach of this present invention will not be interpreted as limited by the above mention modalities that have been described, but that this is determined by a reasonable interpretation of the content of the attached claims.

### BIBLIOGRAPHICAL REFERENCES

[1]. RODRIGUEZ-MARTÍN JOSE MARIA, Electroterapia y Fisioterapia [Electrotherapy and physiotherapy]. Martín Rodríguez 2^{nd} edition, Ed. Panamericana, page 198, 282, y 622.
[2]. DE LOS RIOS-CASTILLO José, BARRIOS-SANTIAGO Pedro, ÁVILA-ROJAS Teresa; ALTERACIONES EMOCIONALES EN PACIENTES DIABÉTICOS CON NEFROPATÍA [Emotional Alterations in Diabetic Patients with Nephropathy]; Jun. 24, 2004, Rev. Med IMSS 2004, page 379-385
[3]. BAKER L L, CHAMBERS R, DeMUTH S K, VILLAR F. Effects of electrical stimulation on wound helling [sic] in patiens [sic] with diabetic ulcers. Department of Biokinesiology and Physical Therapy, University of Southern California, Los Angeles 90033, USA. Diabetes Care. 1997 March; 20(3):405-12.
[4]. GILCREAST D M, STOTTS N A, FROELICHER E S, BAKER L L, MOSS KM. Effect of electrical stimulation on foot skin perfusion in persons with or at risk for diabetic foot ulcers. U.S. Army Nurse Corps, Brooke Army Medical Center, Fort Sam Houston, Tex., USA. Wound Repair Regen. 1998 September-October; 6(5):434-41.
[5]. ARANA VICTORIA, MEJÍA JESÚS, LUENGAS HUMBERTO, SALINAS OLIVIA. Complicaciones del pie diabético, Factores de riesgo asociados. [Diabetic foot complications. Associated risk factors] Rev Med IMSS 2003.
[6]. PETERS E J, LAVERY L A, ARMSTRONG D G, FLEISCHLI J G. Electric stimulation as an adjunct to heal diabetic foot ulcers: a randomized clinical trial. Mexican American Medical Treatment Effectiveness Research Center, Department of Orthopedics, University of Texas Health Sciences Center, San Antonio, Tex., USA. Arch Phys Med/volumen. 2001 June; 82(6):721-5.
[7]. GARZA-ELIZONDO Ma. Eugenia, DIMAS-CALDERÓN Cristóbal, SALINAS-MARTINEZ Ana María, NUÑEZ-ROCHA Georgina, VILLAREAL RIOS Enrique; Atribuciones y creencias sobre la diabetes/volumen/tipo 2 [Features and beliefs about type 2 diabetes]; Jul. 3, 2003, Rev. Med IMSS 2003, page 465-472.
[8]. ROLDAN-VALENZUELA Andrés; Cuidados de Enfermería en el Pie Diabético [Nursing care for diabetic foot]; Registered Nurse; Department of Vascular Surgery; Hospital Universitario Virgen del Rocío de Sevilla; 2001.
[9]. Rev. Farmacología del dolor, fisiologia y tratamiento del dolor [Pharmacology of pain, physiology and treatment of pain] Dr. Susana Tera 23olum [sic], Rev. 1-4. Mex. 2004.
[10]. Medicina Interna, Misael Uribe, 2nd Ed. Panamericana, Page 335, 356, 363, 399.
[11]. Secretos de la Endocrinología [Secrets of Endocrinology], 2^{nd} Ed. McGraw-Hill Interamericana, pag. 40, 41.
[12]. BRAUN B, http://www.ulceras.net/Braun/Ulcerkit.pdf. Manual para el diagnóstico y tratamiento de úlceras [Manual for the diagnosis and treatment of ulcers], Medical S. A, 16 de septiembre del 2003.
[13]. HOUGHTON P E, KINCAID C B, LOVELL M, CAMPBELL K E, KEAST D H, WOODBURY M G, HARRIS K A. Effect of electrical stimulation on chronic leg ulcer size and appearance. School of Physical Therapy, University of Western Ontario, Room 1443, London, Ontario, Canada N6G1H1. Volume 83. Number 1. January 2003.
[14]. JUNGER M, ZUDER D, STEINS A, HAHN M, KLYSCZ T. Treatment of venous ulcers with low frequency pulsed current (Dermapulse): effects on cutaneous microcirculation. Universitats-Hautklinik, Tubingen. Hautarzt. 1997 December; 48(12):897-903. Articulo en alemán.
[15]. LUNDEBERG T C, ERIKSSON S V, MALM M. Electrical nerve stimulation improves healing of diabetic ulcers. Department of Physiology, Karolinska Institutet, Karolinska Hospital, Stockholm, Sweden. Ann Plast Surg. 1992 October; 29(4):328-31.
[16]. COSMO P, SVENSSON H, BORNMYR S, WIKSTROM S O. Effects of transcutaneous nerve stimulation on the microcirculation in chronic leg ulcers. Department of Plastic and Reconstructive Surgery, Malmo University Hospital, Sweden. Scand J Plast Reconstr Surg Hand Surg. 2000 March; 34(1):61-4.
[17]. KUCHERENKO N V, SKRYPOVA T V, LIUTKEVYCH V F, TURANS'KYI A I, SKYBUN V M. Detection and treatment of lower extremity neuropathy in patients with diabetic foot. Klin Khir. 2001 August (8):22-4. Article in Ukrainian.
[18]. ADEGOKE B O, BADMOS K A. Acceleration of pressure ulcer healing in spinal cord injured patients using interrupted direct current. Physiotherapy Department, College of Medicine, University of Ibadan, Ibadan, Nigeria. Afr J Med Med Sci. 2001 September; 30(3):195-7.
[19]. WOOD J M, EVANS P E 3^{RD}, SCHALLREUTER K U, JACOBSON WE, SUFIT R, NEWMAN J, WHITE C, JACOBSON M. A multicenter study on the use of pulsed low-intensity direct current for healing chronic stage II and stage III decubitus ulcers. Department of Dermatology, University of Hamburg, Germany. Arch Dermatol. 1993 August; 129(8):999-1009.
[20]. Terapias de apoyo para el tratam ento de úlceras por presión [Support therapies for the treatment of pressure ulcers]. HEALTH SERVICES/TECHNOLOGY ASSESSMENT TEXT: http://www.ncbi.nlm.nih.gov/entrez/query.fcgi?cmd=Search&db=books&doptcmd1=GenBookHL&term=electroth erapy+ulcers+AND+258108%5Buid %5D&rid=hstat2.section.5433#5438
[21]. ARBOLEDAD B. JOSEFA, MANJÓN B. PILAR. Guía clínica de cuidados de úlceras por presión [Clinical guide to the care of pressure ulcers]. Hospital San Juan de la Cruz de Úbeda. 2003.
[22]. BAKER LUCINDA, PhD; CHAMBERS RICHARD, MD; DeMUTH SHARON, MS; VILLAR FERNANDO Phd. Effects of Electrical Stimulation on Wound Healing in Patients With Diabetic Ulcers. Department of Biokinesiology and Physical Therapy (L.L.B., S.K.D., F.V.), University of Southern California, Los Angeles; and the Ortho/Diabetes Service (R.C.), Rwidth Los Amigos Medical Center, Downey, Calif. March 1997.
[23]. INTERNATIONAL DIABETES FEDERATION AND THE INTERNATIONAL WORKING GROUP ON THE DIABETIC FOOT, Diabetes and Fott Care, Time to Act. 2005.
[24]. MINISTRY OF HEALTH, NATIONAL HEALTH INSTITUTE, National Health Survey 2000, Adult health.

1. A low frequency electrical stimulator device for the prevention and healing of chronic wounds, said device comprising an electrical control board said electrical control board comprising:
i) a microcontroller that generates electrical impulses in the form of a square monopolar wave signal and a paused square monopolar wave signal in at least one output channel at different frequencies, said microcontroller generating different time bases for the application of therapy and also providing to each output channel one of the aforementioned frequencies and signal type according to the therapeutic needs of a patient;
ii) at least one signal conditioning circuit which is sensitive to any current variation provided by the signals from outputs of the microcontroller, the signal conditioning circuit providing two signals 180° out of phase with respect to the microcontroller;
iii) at least one controlling and amplifier circuit powered by a voltage supply constituted by opto-transistors and complementary transistors, of which two are of the NPN type and two more are of the PNP type, in an H bridge configuration; wherein the two signals coming out of the signal conditioning circuit activate the opto-transistors and these opto-transistors activate a branch of the H bridge, and based on the phase of the signal conditioning circuit, the bipolar signals are obtained in its two waveforms on the signal amplifier output;
iv) at least one channel indicator circuit to activate a bicolor light emitting diode that is activated depending on the polarization voltages in one of the microcontroller outputs;
v) an oscillatory circuit to set the speed at which the microcontroller executes the instructions stored in its memory, said oscillatory circuit comprising a crystal whose value may change according to the type of controller or speed required;
vi) a pre-establishing circuit to allow the microcontroller to re-establish its system in a manual form, or an automatic form as the device is turned on; and
vii) a regulated power supply to power the circuit and the low frequency electrical stimulator device for the prevention and healing of chronic wounds.

2. The device of claim 1, in which ranges of the frequency, therapy times and type of signal can be conveniently adjusted according to the type of disease being treated, and the general condition of the patient.

3. The device of claim 1, wherein range of the frequency is selected from the group consisting of 50, 100, 150, 200, and 250 Hz.

4. The device of claim 1, in which the time for the application of therapy can be 5, 10, 15 or 30 minutes.

5. The device of claim 1, in which the microcontroller includes a keyboard and a sound alarm.

6. The device of claim 1, comprising optionally one or all of the following elements: a graphic display, an EEPROM external memory or FLASH memory that allows for pre-defined program storage, device configurations, and a serial interface type RS-232 for communication with a device that supports this protocol.

7. The device of claim 1, in which the conditioning signal circuit comprises opto-transistors and resistors.

8. The device of claim 1, in which the control circuit and the amplifier include, in the transistor output, fuses that are placed in series for the protection of the electrical current levels in case these would go above the levels stipulated in the design or overload; and varistors placed in parallel for the protection of a user.

9. The device of claim 1, in which the controller circuit and the output signal amplifier in conjunction with the signal conditioning circuits comprise at least one power circuit, at least one of which would be in the control board.

10. The device of claim 1, in which the number of potency circuits depends on the number of channels of the device.

11. The device of claim 1, in which the channel indicator circuit provides the user with information about the type of impulse that is being applied, and indicates the activated channels.

12. The device of claim 1, further comprising a current amplifier circuit to activate a sound alarm when the microcontroller sends the command signal.

13. The device of claim 1, in which the channel indicator circuit has an NPN transistor and resistors that configure the transistor as a not logic gate.

14. A treatment method for a patients that has skin ulcers stemming from a degenerative disease; said treatment method comprising the following step:
stimulating sensitive nerve endings to provoke the venous and lymphatic vasoconstrictor reflex and the motor response of flat muscle tissue found in the stimulated area, thus accelerating the drainage of fluids found due to trophism and tissue damage caused by the ulceration;
wherein said stimulation step comprises the applying electrical impulses to an area close to the wound, in alternate sessions of 15 minutes of a square bipolar signal, and 15 minutes of a paused square bipolar signal, at 100 Hz in signal, period and an intensity of maximum 200V.

15. (canceled)

16. The method of claim 14, wherein the method is effective in the skin ulcer treatment caused by disease selected from the group consisting of diabetes, arterial disease, trauma, venous and arterial insufficiency.

17. The method of claim 14, wherein the method is configured to cause one or more selected from the group consisting of:
improving circulation;
improving nutrition of the tissue;
accelerating the growth of granulation tissue;
increasing capillary density; and
increasing partial oxygen pressure;
thereby enhancing the healing rate in the patient with ulcers.

18. The device of claim 1, wherein said electrical control board further comprises one or more selected from the group consisting of an oscillatory circuit, a control circuit, alarm mechanism, a regulated voltage power supply, a pre-establishing circuit and a housing that works as a protective cover of said electrical board, wherein said housing can include additional elements selected from the group consisting of a graphic display, a keyboard, potentiometers, adjustment knobs, voltage suppressors, monophonic outlets, “on and off” switch, fuse and power supply cable.
